(19) United States
(12) Patent Application Publication
(10) Pub. No.: US 2010/0174000 A1
(43) Pub. Date: Jul. 8, 2010

(54) CARRIER IN THE FORM OF OIL-IN-WATER EMULSION NOTABLY INTENDED FOR OPHTHALMIC OR DERMOCOSMETIC USE

(75) Inventors: Christian Sarrazin, Pegomas (FR); Marina Do, Menton (FR); Michele Boix, Canthron (FR)

Correspondence Address: BACHMAN & LAPOINTE, P.C., 900 CHAPEL STREET, SUITE 1201, NEW HAVEN, CT, 06510, US

(73) Assignee: OCTALIA TECHNOLOGIES, Vallauris (FR)

(21) Appl. No.: 12/601,886

(22) PCT Filed: May 23, 2008
(86) PCT No.: PCT/FR08/50899

§ 371 Date: Feb. 11, 2010

(30) Foreign Application Priority Data
May 29, 2007 (FR) 0703796

(51) Int. Cl. A61K 9/107; A61P 17/00; A61K 8/97; A61K 8/06; A61P 27/02

(52) U.S. Cl. 514/772.6; 514/772; 514/772.4; 514/779; 514/781; 514/785

(57) ABSTRACT

An oil-in-water type emulsion contains a very small quantity of surfactant, so that the emulsion is stable, well tolerated and non-irritating.

The emulsion is used as the base for compositions intended to be applied to sensitive tissues of the body of a human or animal, in particular in ophthalmic, dermatological and dermocosmetic compositions.

### BACKGROUND

(1) Field of the Invention

The present invention relates to an oil-in-water type emulsion containing a very small quantity of surfactant, so that the emulsion is stable, well tolerated and non-irritating.

The present invention further relates to the use of such an invention as the base for compositions intended to be applied to sensitive tissues of the body of a human or animal, in particular in ophthalmic, dermatological and dermocosmetic compositions.

(2) Prior Art

An emulsion is the mixture of two non-miscible liquids under the effect of mechanical agitation forming a dispersed system. This dispersion remains as long as agitation continues but once agitation stops the globules coalesce and the liquids separate.

The formulation of an emulsion aims at bringing to the mixture the product or products which will enable or facilitate stabilisation of the system. These products are thus essential to the formulation and preservation in terms of physicochemical stability of an emulsion: they are called emulsifiers, surfactants, dispersers, wetting agents or surfactants and must be incorporated in a relatively large quantity, about 1% to 2% in the field of ophthalmics.

Yet surfactants are known for their irritating characteristics, which greatly limit their use in the fields of ophthalmics and dermocosmetics. Thus, in the context of long-term opthalmologic treatments, for example, the repeated administration of preparations on the corneal surface leads to deleterious, allergenic and toxic effects (in the case treatments of ocular dryness, glaucoma, etc.) which can range from simple irritation to serious tissue deterioration likely to compromise the patient's compliance with the treatment.

Much literature exists that broadly describes the state of corneal cytotoxic effects on in vitro models of known surfactant compounds, in particular polysorbate 80.

Similar adverse effects related to the use of surfactants are also observed in the context of dermal applications.

The technical problem that the present invention seeks to solve thus consists in finding a satisfactory compromise between emulsion stability and tissue tolerance.

To overcome this problem, persons skilled in the art sought to eliminate the use of surfactant compounds in such compositions by stabilising said compositions by the use of thickening agents in particular.

Thus, for example, the European patent application EP 1336404 described a fluid lubricating composition that mimics tear film, which contains no surfactant, and is provided in the form of an emulsion, in which the aqueous phase contains at least one water-soluble viscosity agent and the oil phase, which accounts for 1.5% to 13% by weight of this composition, contains 1% to 6% by weight of at least one liposoluble viscosity agent.

Nevertheless, this composition uses as a thickener a compound such as glycerol monostearate, which is in addition known to persons skilled in the art for its emulsifying properties. This is a compound with the consistency of a semi-solid at room temperature, thus conferring high viscosity on the composition which contains it.

Similarly, the international patent application WO 96/37180 described galenical compositions of use in dermatology and cosmetology of the pseudo-emulsion type which do not include surfactants but which have an effect on the thickening of the lipid and aqueous phases by the use of glycerol monostearate.

In addition, European patent EP 1,275,376 described a gel emulsion consisting of an emulsified oil phase in an aqueous solution of polyvinylpyrrolidone or polyvinyl alcohol or dextran or cellulose derivative. The oil phase is immediately introduced into the previously-gelled aqueous phase, so as to block and/or stabilise the lipid globules formed. The polyvinyl alcohol used is a completely hydrolysed type and the concentrations used are between 0.1% and 10%, and preferentially between 0.3% and 1.5%.

### SUMMARY OF THE INVENTION

A goal of the present invention is thus to provide a carrier of the type mentioned above which incorporates only very small quantities of surfactant while preserving the characteristics generally allowed for traditional emulsions that generally contain more, in particular a small lipid globule size of about 2 μm to 50 μm. The thinnest emulsions favour the bioavailability of active agents in carriers and are more stable during preservation.

To this end, the present invention provides a carrier in oil-in-water (O/W) emulsion form comprising at least one aqueous phase (I) and at least one oil phase (II), said aqueous phase (I) comprising at least one water-soluble surfactant and at least one water-soluble thickener.

According to the invention, the water-soluble surfactant accounts for 0.01% to 0.1% by weight, and preferentially roughly 0.03% by weight of the total weight of the carrier.

The inventive carrier is stable, well tolerated and non-irritating.

Below 0.01% surfactant in the aqueous phase of the inventive carrier, said carrier has the disadvantage of containing an insufficient quantity of surfactant to enable the preparation of an emulsion of optimal stability as well as of a desirable fineness in terms of globule size.

Above 0.1% surfactant in the aqueous phase of the inventive carrier, said inventive carrier has the disadvantage of containing too large a quantity of surfactant which renders it incompatible with satisfactory tissue tolerance, particularly for compositions containing much oil.

Examples of water-soluble surfactants that can be used in the inventive carrier include in particular polysorbates, lecithins, polyethylene glycols and derivatives thereof, polyoxyethylene-40-stearate, sorbitan esters, polyoxyethylene-polyoxypropylene copolymers, and polyvinyl alcohols.

Preferentially, according to the invention, polyvinyl alcohol (PVA) is used, and in a more preferred manner partially hydrolysed polyvinyl alcohol, which is widely used in the fields of pharmaceuticals and cosmetics, is used.

In addition, examples of water-soluble thickeners that can be used in the inventive carrier include acrylic polymers or carbomers, xanthan gum, carrageenans, alginates, cellulose and derivatives of same.

Thus, as a water-soluble thickener a polyacrylic acid can advantageously be used, and preferably a crosslinked polyacrylic acid, such as for example the one marketed by NOVEON under the trade name Carbopol® 980 NF. Such a polymer has the property of considerably increasing the viscosity of the emulsion until the emulsion has, most notably, the consistency of a gel.

Similarly, as a water-soluble thickener a C₁₀-C₃₀ alkyl acrylate/acrylate copolymer can be used, such as for example the one marketed by NOVEON under the trade name PEMULEN®.

The oil phase of the inventive carrier is now described in more detail.

The oil phase of the inventive carrier can contain one or more oils chosen among vegetable oils, mineral oils, silicone oils, synthetic oils and fluorinated oils.

Isopropyl myristate is a specific example of a synthetic oil that can be used according to the present invention.

Vegetable oils that can be used according to the invention include jojoba, avocado, sesame, sunflower, corn, soya, safflower, grape seed, olive, sweet almond, castor bean, moringa, coconut, palm, borage, colza, wheat germ, flax, evening primrose, argan, calendula and cotton oil.

The preferred vegetable oils according to the invention are sweet almond oil and sunflower oil.

Mineral oils of use according to the invention include paraffin oils, preferably liquid paraffin oils.

The inventive carrier can further include one or more compatible adjuvants, which do not modify the characteristics specific to emulsions.

Examples of adjuvants compatible with ophthalmic or dermocosmetic use include preservatives, antioxidants, tonicity agents, chelating agents, buffers, polymers, loads, hydrophilic or lipophilic gelling agents, hydrophilic filters, and odour absorbers.

Of course, persons skilled in the art will take care in choosing the optional adjuvant or adjuvants to be added to the inventive compositions, as well as their concentration, so that the advantageous properties held intrinsically by the inventive compositions are not, or not substantially, altered by the addition envisaged.

In particular, these adjuvants will not harm the properties of the inventive composition, in other words good tolerance and the absence of irritability to the eye or skin, according to the mode of application envisaged.

A preferred tonicity agent is glycerin.

If the inventive carrier contains glycerin as a tonicity agent, its quantity will be advantageously adjusted in order to obtain an osmolality suitable for ocular administration.

Advantageously, the oil phase represents 0.5% to 5% by weight, and preferably approximately 1.5% by weight of the total weight of the carrier.

According to a first specific embodiment of the invention, the oil phase represents 0.5% to 3% by weight, preferably approximately 1.5% by weight of the total weight of the carrier.

If a tonicity agent such as glycerin is added to such a carrier, a carrier is obtained that can be used directly as an ophthalmic product to hydrate the eye (artificial tears) and/or to treat dry eye. Such a carrier can also be used as a base in an ophthalmic composition.

The present invention further relates to an ophthalmic composition provided in the form of an oil-in-water emulsion that comprises:
a carrier in conformity with the first embodiment of the invention, and,
at least one active ingredient of ophthalmic use.

The active ingredient of ophthalmic use can be, for example, selected among antiseptics, antibiotics, antivirals, anti-inflammatories, antiallergics, anti-glaucoma agents, vasoconstrictors, anaesthetics, cicatrisation agents, mydriatics, miotics, tear substitutes, products to treat cataracts, products to treat degenerative retinal disease, and diagnostic products.

The active ingredient of ophthalmic use that can be used in the inventive composition can be water soluble and/or liposoluble.

Examples of liposoluble ophthalmic active ingredients usable in the inventive ophthalmic compositions include vitamin A and cyclosporin A.

An example of a water-soluble ophthalmic active ingredient usable in the inventive ophthalmic composition is vitamin B12, used because of its cicatrisation properties.

Generally speaking, the inventive composition can also contain the typical adjuvants used in the ophthalmic field, insofar as the adjuvant does not alter the properties sought for the inventive ophthalmic composition.

According to a second specific embodiment of the invention, the oil phase represents 1% to 5% by weight of the total weight of the inventive carrier. In this case, the carrier can be used as a base in a cosmetic, dermocosmetic, or dermatological composition, in particular one intended to treat sensitive areas of the face, for example around the eyes.

The present invention further relates to a cosmetic, dermocosmetic or dermatological composition comprising:
a carrier according to the invention comprising 1% to 5% by weight of oil phase compared to the total weight of the carrier, and,
at least one active ingredient of cosmetic and/or dermatological use.

The active ingredient of cosmetic and/or dermatological use can also be water soluble and/or liposoluble.

Examples of active ingredients of dermatological use include corticoids, antibiotics, antifungals, antiseptics, antiparasitics, antiherpetics, anti-acne agents, antipruritics, keratolytics, products to treat psoriasis, and products to treat atopic dermatitis.

Specific examples of water-soluble active ingredients of cosmetic and/or dermocosmetic use usable according to the invention include proteins, amino acids, polyols, urea, sugars and sugar derivatives, water-soluble vitamins, vegetable extracts and hydroxy acids.

A particularly preferred water-soluble vitamin for the inventive cosmetic and/or dermatological composition is vitamin B12, which in addition to its cicatrisation properties has the advantage of giving the inventive composition an aesthetically attractive colour from a cosmetic point of view.

Examples of liposoluble active ingredients of cosmetic and/or dermocosmetic use usable according to the invention include in particular liposoluble UVA and UVB filters and vitamins such as retinol (vitamin A) and derivatives of same and tocopherol (vitamin E) and derivatives of same.

The present invention also relates to a method of preparing a carrier, comprising the following steps:
a) preparation of a first oil-in-water emulsion by introducing into a reactor 0.5% to 5% by weight of at least one oil compared to the total weight of the carrier, then addition of an aqueous solution of surfactant comprising 1.5% by weight of surfactant in a volume such that the volume ratio of the aqueous phase to the oil phase is at least 2:3 and preferentially 3:2,
b) preparation of an aqueous solution of a water-soluble thickener whose concentration will depend on the thickener used and the application in question,
c) introduction of the first emulsion in the aqueous solution of water-soluble thickener, to obtain a second emulsion comprising an aqueous phase and an oil phase, the quantity of the first emulsion introduced in the aqueous solution of water-soluble thickener being such that the water-soluble surfactant represents 0.01% to 0.1% by weight of the carrier.

The surfactant and the water-soluble thickener are such as those described above.

Preferably, into the aqueous solution of thickener is introduced an aqueous solution of a tonicity agent, preferably glycerin, at a concentration of approximately 2.3%. The introduction of this aqueous solution of tonicity agent makes possible an osmolality of roughly 270 mOsm/kg, which is compatible with an ophthalmic application.

### DETAILED DESCRIPTION OF THE PREFERRED EMBODIMENT(S)

The invention will be now illustrated using the following non-limiting examples. The quantities are expressed in weight percent unless otherwise specified.

### Examples

### Products

Components of the Oil Phase
vegetable oil: sunflower oil or sweet almond oil

Components of the Aqueous Phase
purified water
soda
water-soluble surfactants: polyvinyl alcohol (PVA), polysorbate 80
water-soluble thickeners: crosslinked polyacrylic acid, marketed by NOVEON under the trade name CARBOPOL®; alkyl acrylate/acrylate copolymer, marketed by NOVEON under the trade name PEMULEN®
tonicity agent: glycerin

### Example 1

A first example of inventive carrier C1 is prepared as follows.

A first emulsion (O/W) is prepared by dispersion of oil in a volume of 20 ml of aqueous solution of PVA at 1.5% by weight. The introduction of oil into the aqueous solution of PVA is made under strong agitation (Ultra-turrax, 1 minute).

In addition, 23 g of glycerin is introduced into approximately 600 ml of an aqueous solution of alkyl acrylate/acrylate copolymer under magnetic stirring to obtain a monophasic solution of glycerin and alkyl acrylate/acrylate copolymer.

Then, the first PVA-based emulsion (O/W) is introduced in the monophasic solution of glycerin and alkyl acrylate/acrylate copolymer under magnetic stirring to obtain the final emulsion constituting carrier 1 according to the invention. The pH is then adjusted between 6 and 7 with soda and the final weight is adjusted with water.

The inventive composition E1 is presented in table 1 below.

### Example 2

In the same manner as in example 1, a second example E2 of the inventive composition is prepared by replacing the alkyl acrylate/acrylate copolymer by a crosslinked polyacrylic acid.

The composition of the inventive carrier E2 is also presented in table 1 below.

### Comparative Example CE1

A first example of a control composition, similar to that of example E1 but without surfactant, was prepared. In this case, the oil was emulsified beforehand in a fraction of alkyl acrylate/acrylate copolymer solution.

The composition of the control carrier CE1 is presented in table 1 below.

### Comparative Example CE2

A second control composition, corresponding to that of example E2 but without surfactant, was prepared. To be able to prepare this comparative example which contains no surfactant, the oil was emulsified beforehand in a fraction of the crosslinked polyacrylic acid solution.

The composition of control carrier CE2 is also presented in table 1 below.

### Comparative Example CE3

A third control composition corresponding to that of example E1 was prepared but by replacing the PVA solution with a solution of polysorbate 80, so as to obtain a 1% final concentration of surfactant corresponding to the concentration of surfactant traditionally used in known emulsions of the prior art.

The composition of control carrier CE3 is also presented in table 1 below.

### Comparative Example CE4

A fourth control composition corresponding to that of example E1 was prepared but by increasing the concentration of the PVA solution so as to have a 1% final concentration of surfactant corresponding to the concentration of surfactant traditionally used in the emulsions of the prior art.

The composition of control carrier CE4 is also presented in table 1 below.

TABLE 1

| Composition | E1 | E2 | CE1 | CE2 | CE3 | CE4 |
|---|---|---|---|---|---|---|
| (in g/100 g of | (in g/100 g | (in g/100 g | (in g/100 g | (in g/100 g | (in g/100 g | (in g/100 g |
| carrier) | of carrier) | of carrier) | of carrier) | of carrier) | of carrier) | of carrier) |
| PVA | 0.03 | 0.03 | — | — | — | 1 |
| polysorbate 80 | — | — | — | — | 1 | — |
| glycerin | 2.3 | 2.3 | 2.3 | 2.3 | 2.3 | 2.3 |
| crosslinked | — | 0.25 | — | 0.25 | — | — |
| polyacrylic acid | | | | | | |
| alkyl | 0.05 | — | 0.05 | — | 0.05 | 0.05 |
| acrylate/acrylate | | | | | | |
| copolymer | | | | | | |
| oil | sweet almond | sunflower | sunflower | sunflower | sunflower | sunflower |
| | 1.5 | 1.5 | 1.5 | 1.5 | 1.5 | 1.5 |
| purified water | Qs. 100 g | Qs. 100 g | Qs. 100 g | Qs. 100 g | Qs. 100 g | Qs. 100 g |
| NaOH | QS. pH 6-7 | QS. pH 6-7 | QS. pH 6-7 | QS. pH 6-7 | QS. pH 6-7 | QS. pH 6-7 |

For both of the inventive compositions E1 and E2 and all of the controls CE1, CE2, CE3 and CE4 the following characteristics of the emulsion were evaluated:
macroscopic appearance, pH,
osmolality, and,
measurement of the size of the oil globules in the carrier by laser granulometry. The mean volume expressed in μm is reported for each sample.

The behaviour of the emulsion after centrifugation was also evaluated as a provisional test of stability. The samples were centrifuged for 5 minutes at various speeds and then examined visually in order to determine the physical modifications such as creaming (accumulation of lipid globules on the surface) or phase separation (complete separation of the oil and aqueous phases).

Example E1 according to the invention was monitored for extended stability over 6 months at 4° C. and 40° C. and over 18 months at 25° C. The following parameters were studied: macroscopic and microscopic aspects, pH, osmolality, globule size and viscosity (at 25° C.).

The results obtained are presented in table 2 below.

TABLE 2

| | | | | Globule | | |
|---|---|---|---|---|---|---|
| | | | Osmolality | size (mean | Behaviour after | |
| Compositions | Macroscopic appearance | pH | (mOsm/kg) | volume) | centrifugation | Stability |
| E1 | Very white and | 6.6 | 268 | 4.7 μm | + | No significant change after 6 months |
| | homogeneous emulsion | | | | (No change) | at 4° C. and 40° C. for each parameter |
| | | | | | | tested. |
| | | | | | | No significant modification after 18 |
| | | | | | | months at 25° C. for each parameter |
| | | | | | | tested. |
| E2 | Very white and | 6.8 | 275 | 6.3 μm | + | — |
| | homogeneous emulsion | | | | (No change) | |
| CE1 | White and homogeneous | 6.6 | 268 | 27.6 μm | − | — |
| | emulsion | | | | (Significant | |
| | | | | | creaming) | |
| CE2 | White and homogeneous | 5.2 | 267 | 23.5 μm | + | — |
| | emulsion | | | | (No change) | |
| CE3 | Very white, homogeneous | 6.7 | 277 | 2.2 μm | + | — |
| | and opaque emulsion | | | | (No change) | |
| CE4 | Very white, homogeneous | 6.5 | 281 | 2.9 μm | + | — |
| | and opaque emulsion | | | | (No change) | |

Under the inventive conditions of preparation, examples E1 and E2 containing a small quantity of surfactant (0.03% PVA) have a lipid globule size:
of the same order of magnitude as comparative examples CE3 and CE4 obtained with a quantity of surfactant of 1%, and,
much lower than that obtained without surfactant (comparative examples CE1 and CE2).

In addition, the evaluation of stability after centrifugation reveals the absence of physical changes to the inventive examples E1 and E2 although they contain little surfactant. The absence of surfactant under similar conditions (comparative example CE1) led to creaming of the emulsion.

Comparative example CE2, which does not contain surfactant, did not show physical changes during centrifugation, which is explained by a higher viscosity of this formula.

Inventive example E1 monitored for 6 months at 4° C. and 40° C. and 18 months at 25° C. exhibited satisfactory stability for each parameter studied.

A study of animal ocular tolerance (albino rabbit) of 4 days with dosing schedules up to 6 infusions per day was carried out on preparations corresponding to examples E1 and E2 compared to a control condition represented by physiological saline solution. Ocular examination was carried out using an opthalmoscope and ocular tolerance was evaluated by using the Draize test rating scale as described in the scientific publication “Methods for the study of irritation and toxicity of substances applied topically to the skin and mucous membranes” by Draize et al. (1944), in J. Pharmacol Exper Ther. 82:377.390. Examples E1 and E2 were tolerated equally as well as the control condition (physiological saline solution).

1-20. (canceled)

21. A carrier in oil-in-water (O/W) emulsion form comprising at least one aqueous phase and at least one oil phase, said at least one aqueous phase comprising at least one water-soluble surfactant and at least one thickener, wherein the water-soluble surfactant represents 0.01% to 0.1% by weight of a total weight of the carrier.

22. The carrier according to claim 21, wherein the water-soluble surfactant represents 0.03% by weight of the total weight of the carrier.

23. The carrier according to claim 21, wherein the at least one oil phase is provided in the form of lipid globules in dispersion in the aqueous phase and whose size is equal to or less than 40 μm.

24. The carrier according to claim 21, wherein the water-soluble surfactant is selected from the group consisting of polysorbates, lecithins, polyethylene glycols and derivatives thereof, polyoxyethylene-40-stearate, sorbitan esters, polyoxyethylene-polyoxypropylene copolymers, and polyvinyl alcohols.

25. The carrier according to claim 24, wherein the water-soluble surfactant is a polyvinyl alcohol (PVA).

26. The carrier according to claim 25, wherein the polyvinyl alcohol (PVA) is partially hydrolysed.

27. The carrier according to claim 21, wherein the at least one thickener a water-soluble thickener selected from acrylic polymers, xanthan gum, carrageenans, alginates, cellulose and derivatives of same.

28. The carrier according to claim 27, wherein the water-soluble thickener is a polyacrylic acid.

29. The carrier according to claim 28, wherein the polyacrylic acid is a crosslinked polyacrylic acid.

30. The carrier according to claim 27, wherein the water-soluble thickener is a C₁₀-C₃₀ alkyl acrylate/acrylate copolymer.

31. The carrier according to claim 21, wherein the at least one oil phase comprises at least one oil chosen from the group consisting of mineral oils, vegetable oils, silicone oils, synthetic oils and fluorinated oils.

32. The carrier according to claim 31, wherein the at least one oil is a vegetable oil selected from jojoba, avocado, sesame, sunflower, corn, soya, safflower, grape seed, olive, sweet almond, castor bean, moringa, coconut, palm, borage, colza, wheat germ, flax, evening primrose, argan, calendula and cotton oil.

33. The carrier according to claim 21, wherein the at least one oil phase represents 0.5% to 5% by weight.

34. The carrier according to claim 33, wherein the carrier includes at least one adjuvant selected from the group consisting of preservatives, antioxidants, tonicity agents, chelating agents, buffers, polymers, loads, hydrophilic or lipophilic gelling agents, hydrophilic filters, and odour absorbers.

35. The carrier according to claim 34, wherein the carrier includes a tonicity agent and the at least one oil phase represents 0.5% to 3% by weight of the total weight of the carrier, so that said carrier is compatible with an ophthalmic use.

36. The carrier according to claim 35, wherein the tonicity agent is glycerine and the at least one oil phase represents approximately 1.5% by weight of the total weight of the carrier.

37. The carrier according to claim 34, wherein the at least one oil phase represents 1% to 5% by weight, preferably approximately 3% by weight of the total weight of the carrier, so that said carrier is compatible with one of a cosmetic use and a dermatological use.

38. The carrier according to claim 37, wherein the at least one oil phase represents approximately 3% by weight of the total weight of the carrier.

39. An ophthalmic composition provided in the form of an oil-in-water emulsion, said composition comprising the carrier according to claim 35, and at least one active ingredient for ophthalmic use.

40. A cosmetic and/or dermatological composition provided in the form of an oil-in-water emulsion, said composition comprising a carrier according to claim 39 and at least one active ingredient for cosmetic and/or dermatological use.

41. A method of preparing a carrier such as defined according to claim 21, comprising the steps of:
a) preparing a first oil-in-water emulsion by introducing into a reactor from 0.5% to 5% by weight of at least one oil compared to a total weight of the carrier, then adding an aqueous solution of surfactant comprising 1.5% by weight of surfactant in a volume such that a volume ratio of an aqueous phase to an oil phase is at least 2:3;
b) preparing an aqueous solution of a water-soluble thickener whose concentration depends on the thickener and an application in question; and
c) introducing the first emulsion in the aqueous solution of water-soluble thickener to obtain a second emulsion comprising the aqueous phase and the oil phase, wherein the quantity of the first emulsion introduced in the aqueous solution of water-soluble thickener is such that the water-soluble surfactant represents 0.01% to 0.1% by weight of the carrier.

42. The method according to claim 41, wherein the water-soluble surfactant is polyvinyl alcohol and the water-soluble thickener is an acrylic polymer.

43. The method according to claim 41, further comprising before step c), introducing an aqueous solution of tonicity agent in an aqueous solution of water-soluble thickener.

44. The method according to claim 41, wherein said tonicity agent introducing step comprises introducing glycerine.

45. The method according to claim 41, wherein said adding step forms a volume ratio of 3:2.
